(19) United States
(12) Patent Application Publication
(10) Pub. No.: US 2006/0280700 A1
(43) Pub. Date: Dec. 14, 2006

(54) Oral hygiene system to fight the effects of aging on the mouth, gums, and teeth

(76) Inventor: Stuart L. Isler, Denville, NJ (US)

Correspondence Address: ERNEST D. BUFF;ERNEST D. BUFF AND ASSOCIATES, LLC., 231 SOMERVILLE ROAD, BEDMINSTER, NJ, 07921, US

(21) Appl. No.: 11/148,037

(22) Filed: Jun. 8, 2005

(51) Int. Cl. A61K 8/97; A61K 36/71; A61K 36/82; A61K 8/22

(52) U.S. Cl. 424/53; 424/58; 424/726; 424/729

(57) ABSTRACT

A comprehensive system prevents age related damage to oral tissue and teeth. The system comprises a Daytime composition, a Nighttime composition and an Overnight gel, each applied to mouth, gums and teeth on a daily basis. The Daytime and Nighttime compositions are applied by a special applicator with a brush like tip using the ‘click’ of a ratchet mechanism that delivers a precise quantity of the composition. The Daytime and Nighttime compositions have active ingredients of xylitol and menthol. The Overnight gel is delivered between the tunnels of the teeth by a graduated syringe with a tapered tip. The Overnight gel composition has active ingredients of eucalyptol.

### BACKGROUND OF THE INVENTION

1. Field of the Invention

The present invention relates to a set of oral hygiene compositions designed to combat the effects of aging of oral tissue including mouth, gums, and teeth; and a system for applying these oral hygiene compositions to provide optimal benefit.

2. Description of the Prior Art

Many patents address issues related to oral care and polishing of teeth enamel. The patents and publications that relate to compositions are discussed first.

U.S. Pat. No. 4,332,791 to Raaf et al. discloses a toothpaste active against plaque comprising a copper compound and a silica-polishing agent. The '791 patent relates to a toothpaste which includes a silica polishing agent and at least one copper compound. The '791 patent claims that the subject toothpaste has proven effective against plaque formation on teeth while not exhibiting any of the side effects of the prior art formulations such as discoloring. The silica-polishing agent polishes teeth while the copper compound displays anti-microbial activity. Copper compounds are generally toxic. This toothpaste composition does not protect gum, teeth and oral tissue from age related damage.

U.S. Pat. Nos. 5,925,335, 6,207,137, and 6,503,483, and US Patent publication No. 2002/0054857 to Shuch et al. disclose an orally absorbable improved dental formulation. The dental formulation includes a base to which active components are added. The active components comprise, based on the overall weight thereof, Vitamin C in an amount between about 0 to 25 wt % or 10 to 25 wt %, and coenzyme Q-10 (or ubiquinone), in an amount between 0 to 25 wt % or 10 to 25 wt %, which are added to the base. The dental formulations of the aforementioned references are primarily intended for preventing gum disease and tooth decay, as well as cleaning the teeth and freshening the breath. This vitamin C rich and coenzyme rich composition is used only during brushing of teeth and is not a system designed to protect gum, teeth and oral tissue from age related damage.

U.S. Pat. No. 6,379,652 to Liu et al. discloses a method to suppress oral malodor and provide long lasting breath protection. An oral composition is applied to the oral cavity of the user comprising an orally acceptable vehicle containing therein a flavor system. The flavor system is comprised of a mixture of an essential oil and a coolant compound which is a menthyl ester of naturally occurring hydrocarboxylic acids having 2 to 6 carbon atoms esterified with C₁-C₄ alkyl groups. The oral compositions of the '652 patent are primarily intended for suppressing oral malador and for providing long lasting breath protection. This oral malador suppressing composition does not protect gum, teeth and oral tissue from age related damage.

U.S. Pat. No. 6,723,304 to Stier discloses oral care compositions such as toothpaste, gels, tooth powders, mouthwashes, mouth rinses, gums, mouth sprays and lozenges comprising diglycerol. The diglycerol is used as a humectant in the compositions.

The compositions may further comprise water, flavoring agents, active compounds, emulsifier, alcohol, sweeteners, thickening agents, surfactants, suspending agents, astringent and toning drug extracts, flavor correctants, abrasives or polishes, deodorizing agents, preservatives, flavoring buffers, whitening agents, wound-healing and inflammation inhibiting substances, colorants, dyes, pigments, abrasives, polishes, antimicrobial agents, pH buffers and other additives and fillers. The oral care compositions of the '304 patent are primarily intended for providing an enhanced and/or prolonged smooth lasting effect such as long lasting flavor and cooling characteristics in the mouth. This oral care composition does not protect gum, teeth and oral tissue from age related damage.

US Patent Application Publication No. 2003/003059 to Dana discloses oral care compositions which provide a means to deliver actives which are useful in the prevention, treatment and/or management of dental and related tissue conditions, including dental caries, dental cavities, microbial flora, tartar, periodontal and related gum disease. The composition of the '059 invention can be used in the healthy maintenance of teeth and gums, to whiten teeth and favorably impact the cosmetic appeal of the teeth and gums of a patient. The inclusion of effective amounts of colostrum in dental care compositions has unexpectedly high efficacy in inhibiting, reducing or otherwise preventing microbial growth, dental caries, plaque, cavities and gum disease, including periodontal disease. The use of colustrum with other enzymes as described herein represents a particularly preferred embodiment of the '059 invention because of the unexpected antimicrobial activity exhibited by the enzyme combination. This antimicrobial composition does not protect gum, teeth and oral tissue from age related damage.

U.S. Patent Application Publication No. 2003/0219390 to Santarpia, III et al. discloses an aqueous tooth whitening liquid composition comprising an orally acceptable vehicle of water and monohydric alcohol having dispersed therein a film forming combination of a poly(ethylene oxide) and a Carbomer. The pH of the composition is adjusted to be at an acid value. The liquid whitening composition may also contain a flavoring agent which includes essential oils as well as various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Of these, the most commonly employed are the oils of peppermint, spearmint and wintergreen. The flavoring agent is incorporated in the whitening liquid composition at a concentration of about 0.1 to about 2% by weight and preferably about 0.1 to about 0.5% by weight. The tooth whitening composition of the '390 patent application is primarily intended for whitening the teeth. This whitening composition does not protect gum, teeth and oral tissue from age related damage.

US Patent Application Publication No. 2003/0232023 to Arnold discloses an oral hygiene system, which includes a whitening solution, and applicator, and a rinse. The whitening solution includes a concentration of hydrogen peroxide for whitening teeth within a predetermined period of time without the use of a mouthpiece. The applicator may include a resilient head capable of conforming to the topography of a tooth surface, and may be used for applying gelatinous whitening solution to teeth. The rinse is configured to reduce organic sulfides in the mouth, and may include zinc acetate. The whitening solution may be applied to the teeth for predetermined amount of time, for example, less than about 10 minutes. The solution may then be rinsed off with the rinse. The procedure may then be repeated periodically, for example, daily until the desired shade of whiteness is achieved. This whitening composition does not protect gum, teeth and oral tissue from age related damage.

US Patent Application Publication No. 2004/0091432 to Dulin discloses a portable medication delivery system for treatment of bad breath, gingivitis, ANUG, plaque, etc., comprising small, shirt pocket-sized re-closable pouches having 2 to 4 sterile, single-use, disposable, treated cotton rolls carrying a selected periodontal anti-microbial, such as mouthwash, or a fluoride, enzyme or antibiotic composition. The anti-microbial is preferably a neutral, alcohol-free solution, but may be a dry or gel composition. The treated rolls are placed in the upper and/or lower buccal vestibules where they release, over time, the anti-microbial solution. The treatment directly targets the critical areas, tiny “sulcus” grooves and spaces between the teeth, both significant reservoirs for bacteria. The rolls are left in place for extended periods, on the order of 5-30 minutes or more, without discomfort and without interfering with normal activities of speaking or travel. Tests show the medicated rolls are effective in treatment of bad breath. The oral hygiene system of the '432 patent application is primarily intended for the treatment of bad breath, gingivitis, ANUG, and plaque. This system is a short-term solution for bad breath and does not protect gum,. teeth and oral tissue from age related damage.

US Patent Application Publication No. 2004/0115247 to Melman discloses dental compositions and methods for preventing dental plaque and carie formation and generally for inhibiting tooth decay and brightening/whitening teeth. The compositions comprise organic acids such as acetic acid and salts thereof which can be combined with pharmaceutically acceptable carriers or diluents to be administered in the form of conventional dental compositions. The compositions also preferably contain sodium hexametaphosphate. In a preferred embodiment, the composition is coated on or included in a dental treat, chew or toy for canines. This is a dental plaque control system for dogs and has nothing to do with protecting gum, teeth and oral tissue from age related damage of humans.

Foreign Patent No. CA 2297454 to Wuelknitz et al. discloses oral and dental hygiene products consisting of aqueous, aqueous-alcoholic or anhydrous preparations containing an active combination of a water-soluble potassium or strontium salt and eugenol or oil of cloves, for desensitizing sensitive teeth. The combination contains preferably at least 0.5 wt. % potassium or strontium ions, e.g. in the form of the nitrate, and at least 0.01 wt. % eugenol, preferably in the form of oil of cloves. Desensitizing toothpastes contain an additional 5-50 wt. % of a polishing agent, 1 wt. % thereof being dicalcium phosphate dihydrate and 10-60 wt. % being a humectant, and preferably 0.1-0.5 wt. % magnesium in the form of a dissolved salt and 0.2-2 wt. % monofluorophosphate. The oral products of the '454 patent are primarily intended for desensitizing sensitive teeth. This desensitizing composition does not protect gum, teeth and oral tissue from age related damage.

Foreign Patent No. KR 2000016351 to Iyer et al. discloses oral hygiene compositions which include an antimicrobial agent selected from cedarwood oil, chloramphenicol, citronella oil, Glycyrrhiza glabra extract, juicy fruit basil oil, lemon basil oil, and Rosmarinus officinalis oil. Application of these oral hygiene compositions to the oral cavity effectively reduces or prevents the growth of bacteria associated with dental plaque, and with dental caries and/or periodontal diseases such as Actinomyces viscosus, Campylobacter rectus, Fusobacterium nucleatum, Porphyromonas gingivalis, Streptococcus mutans, and Streptococcus sanguis. The oral products of the '351 patent are primarily intended for preventing plaque, dental caries and periodontal diseases. This antimicrobial composition does not protect gum, teeth and oral tissue from age related damage.

Published Literature Article at J Clin Periodontol. 1992 Sep.; 19(8): 578-82 at PMID: 1447382 discloses a comparison of natural product, triclosan and chlorhexidine mouth rinses on 4-day plaque growth. The influence of other rinse components were assessed, notably sodium lauryl sulphate (SLS) and zinc. The 0.2% chlorhexidine rinse (positive control) was significantly more effective, and the saline rinse (negative control) significantly less effective, than other rinses. Sanguinarine alone was little different from saline and the addition of zinc made a modest improvement in activity. The 3 triclosan/SLS rinses were more effective than the sanguinarine rinses but similar to their minus triclosan control rinse. A natural product/SLS experimental rinse was second to chlorhexidine and, in many analyses, significantly better than all other rinses, but caused some oral erosions. The results indicate that the plaque inhibitory properties of basic ingredients such as SLS may be difficult to enhance or surpass. However, the possible range of recipes, particularly using natural ingredients, provides scope for research and development in the field of oral hygiene products. The oral rinse compositions are primarily intended for preventing plaque formation and do not protect gum, teeth and oral tissue from age related damage.

Published Literature Article at J Clin Periodontol 1997 Sep.;24(9 Pt 1):636-9 at PMID: 9378835 discloses a 4-day plaque regrowth study comparing an essential oil mouth rinse with a triclosan mouth rinse. The essential oil rinse produced a plaque reduction of 52% for plaque area and 17% for plaque index compared to its placebo, whilst the triclosan rinse produced a 45% reduction for plaque area and 12% reduction for plaque index against its placebo. The essential oil rinse significantly reduced plaque compared to the triclosan rinse for plaque index but not for plaque area. The findings of this study would suggest that of the 2 rinses, the essential oil rinse would be expected to be more effective at reducing plaque formation in the longer term. The essential oil rinse composition is primarily intended for preventing plaque formation and does not protect gum, teeth and oral tissue from age related damage.

Published Literature Article at J Am Dent Assoc. 2003 Mar;134(3):359-65 at PMID: 12699051 discloses the efficacy of an essential oil antiseptic mouth rinse vs. dental floss in controlling interproximal gingivitis: a comparative study. The use of dental floss has long been considered to be effective in controlling interproximal plaque and gingivitis.

The authors compared this method with that of use of a mouth rinse. The essential oil-containing mouth rinse group and the brushing and flossing group had significantly lower (P<0.001) mean interproximal Modified Gingival Index, or MGI, scores. In conjunction with professional care (prophylaxis) and tooth brushing over six months, rinsing twice daily with an essential oil-containing mouth rinse was at least as good as flossing daily in reducing interproximal plaque and gingivitis. The brushing and essential oil mouth rinse or flossing alone is insufficient to protect gum, teeth and oral tissue from age related damage.

Published Literature Article at Oral Microbiol Immunol. 2003 Dec.;18(6):389-92 at PMID: 14622345 discloses the susceptibility of oral bacteria to Melaleuca alternifolia (tea tree) oil in vitro. The in vitro activity of Melaleuca alternifolia (tea tree) oil against 161 isolates of oral bacteria from 15 genera was determined. Minimum inhibitory concentrations (MIC) and minimum bactericidal concentrations (MBC) ranged from 0.003 to 2.0% (v/v). MIC90 values were 1.0% (v/v) for Actinomyces spp., Lactobacillus spp., Streptococcus mitis and Streptococcus sanguis, and 0.1% (v/v) for Prevotella spp. Isolates of Porphyromonas, Prevotella and Veillonella had the lowest MICs and MBCs, and isolates of Streptococcus, Fusobacterium and Lactobacillus had the highest. Time kill studies with Streptococcus mutans and Lactobacillus rhamnosus showed that treatment with >or =0.5% tea tree oil caused decreases in viability of >3 log colony forming units/ml after only 30 s, and viable organisms were not detected after 5 min. These studies indicate that a range of oral bacteria are susceptible to tea tree oil, suggesting that tea tree oil may be of use in oral healthcare products and in the maintenance of oral hygiene. Tea tree oil alone is insufficient to protect gum, teeth and oral tissue from age related damage.

The patents which disclose an oral compositions applicator are discussed next.

U.S. Pat. No. 5,456,672 to Diederich et al., discloses a syringe-like device for the dosing of liquids or pastes. A liquid can be dosed advantageously using a syringe-like device, from a container fitted thereon, by means of a plunger mounted displaceably in a dosing chamber, this dosing chamber being connected to the container via a channel passing through this plunger, a valve and a channel arranged together with the plunger in a connection tube. The '672 device does not deliver metered quantities of a composition between the tunnels of teeth and has a needle-like channel extending from the tip of the syringe, not a brush-like tip. The device uses a valve to create a vacuum to draw in the liquid or paste, and stores it in the device until it is time to release the contents.

U.S. Pat. No. 5,611,687 to Wagner, discloses an oral hygiene delivery system. It discloses a delivery system for a liquid oral hygiene preparation suitable for tooth whitening, tooth cleansing and the treatment of plaque. The delivery system includes an elongated barrel shaped body. A supply of the hygiene preparation saturates a fibrous wadding carried in a hollow chamber of the body. At an end of the body, an applicator formed of felt or synthetic fibers is seated. The applicator includes a broad tip and a stem wick, which is received in the wadding and draws the preparation to the tip by capillary action. The preparation is applied to tooth surfaces, oral lesions, and the like by pressing the tip against the surface to receive the preparation and, where appropriate, wiping the tip along the surface. In an alternate embodiment, a ball applicator is provided and the hygienic preparation may be carried in the chamber without the wadding. The requirement that the composition is saturated in the fibrous wading limits the type of compositions that can be used. The tip of the '687 device must be pressed against the tooth surface to receive the preparation, thereby preventing application of controlled amounts. The applicator tip is not removably mounted to the end of a syringe.

U.S. Pat. No. 6,082,999 to Tcherny et al. discloses an interdental device and container. It comprises a plastic dental article, including an outer container made up of an outer shell and a tubular insert. It also includes a stem having an interdental pick and a brush near the pick but spaced slightly therefrom. The container is non-spill, when turned over, either when the stem is in place therein, or removed therefrom. The '999 device is housed within a container having a quantity of dental liquid therein, which saturates the tip of the device. Therefore, known quantity of the liquid composition is not deliverable at any time.

U.S. Pat. No. 6,202,897 to Martin et al discloses a unit dose liquid dispensing and packaging for dental application. It discloses a nozzle for storage and application of liquid dental material, such as primer or adhesive, to a preparation site at a patient's teeth, gums or mouth. The nozzle includes a hollow nozzle for containing the liquid dental material. The hollow nozzle can include a flocked, open tip through which the liquid dental material is dispensed. The nozzle also includes a storage block having a plurality of cavities formed therein. A plurality of hollow nozzles can be inserted into the cavities to form substantially airtight seals between the open tips of the hollow nozzles and the exterior of the cavities. An end of the hollow nozzles opposite from the open tip and exposed above the block includes a breakable seal. Accordingly, the hollow nozzles can be stored in the block for an extended period of time. To apply the liquid dental material to a preparation site, an applicator can be attached to the end of the hollow nozzle exposed above the block. The applicator is adapted to break the breakable seal and force the liquid dental material from the interior of the hollow nozzle through the open tip and onto an application site. The '897 device has a tip that is spherical, not a brush-like tip and cannot deliver liquid between the tunnels of teeth. The measured composition is delivered to saturate the spherical tip but is not delivered to the teeth.

U.S. Pat. No. 6,206,192 to Winstead et al. discloses a dental emergency kit. It discloses a kit for the emergency treatment of dental conditions. The kit includes a container and a plurality of dental treatment components positioned within the container, the dental treatment components being selected from the group consisting of dental cement or components thereof, an anesthetic, an antiseptic, sorbent material for wrapping around teeth to be treated or for packing into apertures of the teeth to be treated such as resulting from broken teeth, missing teeth, missing or broken fillings, or crowns or bridges, and tools for manipulating the dental cement and the sorbent material. This emergency treatment kit has nothing to do with to protecting gum, teeth and oral tissue from age related damage.

U.S. Pat. No. 6,413,087 to Petrich et al. discloses a packaged applicator assembly that includes an applicator having a tip and a cap initially extending over the tip and detachably connected to the applicator. The applicator includes a flexible portion that can be bent in an arc as the cap is detached from the applicator in order to enhance access to the application site when needed. In preferred embodiments, the tip is supplied with a quantity of composition such as a dental composition, and as a result is especially advantageous for single patient use. The '087 device has a spherical tip extending from the applicator, not a brush-like tip, uses a compressible porous material within the cap to store and dispense the composition to the spherical tip of the device. Precise quantity of the composition is therefore not delivered to oral tissue to protect gum, teeth and oral tissue from age related damage.

U.S. Pat. No. 6,450,810 to Fisher et al. discloses cushioned, fiber-covered dental applicators. It discloses a dental instrument which is configured to be positioned adjacent the teeth and gums of a patient without injuring or causing pain to sensitive teeth or gums. The dental instrument includes: (i) a main body having a proximal end and a distal end, the main body being configured to be grasped by a practitioner; (ii) an elastomeric member coupled to the distal end of the main body such that abutment of the elastomeric member against the teeth or gums of a patient does not injure or cause pain to the teeth or gums; and (iii) a plurality of fibers disposed on the elastomeric member. The fibers can be used to remove air bubbles from impression material, or for a variety of other uses. The instrument may be in the form of an applicator or delivery tip, for example. The instrument can have a non-slip gripping surface.

There remains a need in the art for compositions and an application system that delivers predetermined quantities of the compositions to oral tissue including the mouth, gums and teeth in order to combat the effects of age related oral degradation.

### SUMMARY OF THE INVENTION

The present invention discloses a system that provides daily care to help its users achieve a more youthful, healthy smile. It comprises three separate topical compositions: (i) a daytime whitener, moisturizer, antioxidant/anti-inflammatory, and plaque remover; (ii) a nighttime whitener, moisturizer, antioxidant/anti-inflammatory and plaque remover; and (iii) an overnight power gel. The system also includes a device which delivers predetermined quantities of the daytime whitener and nighttime whitener compositions to the mouth, gums and teeth and is used twice on a daily basis, thereby protecting the oral tissues from age related damage. The overnight power gel composition is applied using a device separate from that which delivers the daytime composition and nighttime composition.

Generally stated, the system comprises three discrete topical compositions, each having combinations of ingredients which are absorbed by the oral tissue, thereby preventing or minimizing the effects of aging on the mouth, teeth and gums. Each of the three topical compositions comprises certain active ingredients, effective herbal ingredients and inert ingredients.

As used herein, the term “active ingredients” or “active components” means ingredients which function as a germicidal and/or an anticaries agent in the mouth, gums or teeth. The term “effective herbal ingredients” or “effective herbal compounds” means ingredients from natural sources which function and have effects as an antimicrobial and/or antioxidant/anti-inflammatory agent in the mouth, gums and teeth. The compositions are tailored to be most effective for use during daytime, nighttime and overnight use respectively. The term “inert ingredients” or “inert components”, as used herein, refers to those ingredients in the compositions which are tailored to enhance dispersion of the components of the compositions, as well as enhancing the viscosity and delivery characteristics of the compositions and in maintaining a protective buffering capacity which mimicks the physiological pH of the oral tissues. For example, the daytime and nighttime compositions are free flowing so that each is distributed uniformly over the oral tissue. On the other hand, the overnight gel composition is a gel, which remains undisturbed on the gum, mouth and teeth surfaces while the user is sleeping, providing enhanced contact and absorption time.

The active components of the daytime and nighttime compositions, respectively, comprise xylitol and menthol. Xylitol has anticaries or cavity preventing properties and inhibits the growth of caries- inducing bacteria. Replacement of sucrose and other products with xylitol prevents the growth of Streptococcus mutans bacteria, known to be a major contributor to the formation of dental cavities. The mutans act on sugar molecules creating acid, which has cavity forming ability. Xylitol, on the other hand, suppresses the acid formation capability of mutans bacteria and also prevents the growth of the bacteria, thereby providing an improved bacteria-free environment within the oral tissue including mouth, gums, teeth and oral tissue. Menthol chemically is an aromatic secondary alcohol obtained from the official oil of peppermint. Menthol possesses the general properties of peppermint oil somewhat intensified. It has germicidal, antiseptic and pronounced tonic properties. It has mild local anesthetic properties and counterirritant qualities. As a component of the daytime and nighttime compositions, it provides the soothing properties, and the mild germicidal actions to compliment the anticaries action of xylitol.

The effective herbal ingredients of the daytime and nighttime compositions, respectively, comprise green tea extract and goldenseal extract. The green tea extract blocks the production of oral bacteria associated with periodontal disease. Moreover, green tea extract has strong antioxidant properties and decreases oxidative DNA damage. Goldenseal extract has anti-inflammatory properties and has anti-microbial properties against pathogens such as streptococcus mutans and fusobacterium nucleatum. These effective herbal ingredients in combination have an enhanced effect on reducing the microbial activity within the oral tissue and have healing properties that decrease the incidence of inflammation and lesions within the oral tissues.

The inert ingredients in the daytime and nighttime compositions comprise: taste and flavor enhancers including spearmint oil, stevia extract (a sugar free sweetener); mineral replacement additions including calcium chloride dehydrate, magnesium chloride; a buffer-system to maintain physiological pH including potassium phosphate monobasic, potassium phosphate dibasic, potassium chloride; bleaching or whitening agent including urea hydrogen peroxide; moisturizers including Arsalene 19, and thickeners including sodium carboxymethylcellulose and preserved water. The taste flavor enhancers improve the taste and flavor of the compositions. The mineral replacement additions provide an environment that strengthens the enamel and reduces its tendency to form cavities, and prevents discoloration and damage. The buffer-system is an acid neutralizer which maintains the physiological pH to prevent acidic damage to the oral tissues. The bleaching/whitening agent essentially works against harmful microbes present and removes surface stains on hard tissue. The thickeners adjust the viscosity so that the compositions can be easily livered through the device and into the “tunnels” between the teeth and gum line.

The daytime composition has from 0.025% to 0.9% active ingredients with menthol and xylitol in a ratio of 1:36. The effective herbal components including green tea extract and goldenseal are included as a 0.06% concentration each. The inert ingredients include flavor enhancers, mineralizers, buffers, bleaching/whitening agent, and viscosity modifiers ranging from 0.006% to 5.000%.

The nighttime composition has from 0.025% to 0.9% active ingredients with menthol and xylitol in a ratio of 1:36. The effective herbal components including green tea extract and goldenseal are included as a 0.06% concentration each. The inert ingredients include flavor enhancers, mineralizers, buffers, bleaching/whitening agent, and viscosity modifiers ranging from 0.006% to 10.000%.

The active component of the Overnight gel composition comprises the biologically active extract of eucalyptus (eucalyptol). Eucalyptol has antibacterial effects against a number of different periodontopathic bacteria. It also has anti-inflammatory and analgesic (pain relieving) properties. The effective herbal ingredients of the Overnight gel composition comprise Aloe Vera and Melissa officinalis (Lemon Balm) Oil. Aloe Vera extract is an effective tissue moisturizer and is an anti-inflammatory. It further has the ability to stop dental bleeding. Melissa officinalis (Lemon Balm) oil is an effective anti-oxidant against selected oxidants, which could damage oral tissue. Topical application of Melissa is useful as prophylaxis (a preventative) against periodontal disease.

The inert components of the Overnight gel include flavor and taste enhancing ingredients including Spearmint Oil, Stevia Extract (a non sucrose sweetener), Carbopol 934P NF thickener, a moisturizer retainer Trolamine NF (Triethanolamine) and Preserved Water.

The Overnight gel composition has the active component eucalyptol in concentration of 1.000%. Effective herbal ingredients Aloe Vera and Melissa Officinalis (Lemon Balm) Oil) are included in concentrations of 0.500% and 0.01% respectively in ratio of 50:1. The inert ingredients include flavor enhancers, viscosity modifiers and moisture retainers ranging from 0.500% to 1.5%.

A set of Applicators are especially tailored to apply the Daytime Composition and the Nighttime Composition, respectively. The Overnight Gel is applied using a graduated syringe with a tapered applicator tip that fits into the tunnels between the teeth, that is, the gaps between the gum line and teeth surface. The Daytime composition applicator, Nighttime composition applicator and the Overnight Gel are organized in a time efficient and reliable manner in the system kit. The applicators are especially tailored for use in applying the compositions to help fight the effects of aging on the mouth, gums and teeth and to remove plaque on a round-the-clock basis. When used in connection with the present System, the applicators facilitate application of the oral hygiene products, providing twenty-four, seven protection against plaque formation and the effects of aging on the mouth, gums and teeth.

The key features of the oral hygiene system to fight the effects of aging on the mouth gums and teeth include, in combination, the features set forth below:
1. The system comprising three topical compositional elements;
2. The first composition is a Daytime composition comprising active ingredients xylitol and menthol, together with effective herbal ingredients green tea extract and goldenseal extract together with inert ingredients that enhance the flavor and taste of the composition, a thickener to adjust the viscosity of the composition and a teeth whitener;
3. The second composition is a Nighttime composition comprising active ingredients xylitol and menthol, together with effective herbal ingredients green tea extract and goldenseal extract together with inert ingredients that enhance the flavor and taste of the composition, a thickener to adjust the viscosity of the composition and a larger quantity of teeth whitener;
4. The third composition is an Overnight gel comprising the active ingredient eucalyptol, the effective herbal ingredients aloe vera and Melissa officinalis (Lemon Balm) Oil together with the inert ingredients that enhance the flavor and taste of the composition, a thickener'to adjust the viscosity of the composition and moisture retainer;
5. An applicator is provided with a brush like tip capable of delivering Daytime and Nighttime compositions to the tunnels between teeth and at the gum-teeth line from a barrel through the use of a ratchet mechanism that delivers a precise amount of the composition per click of the ratchet, wherein daily application requires approximately 20 clicks;
6. A graduated syringe applicator with a tapered tip is provided for delivering the Overnight gel; whereby the overall health of the oral tissues including mouth, gums and teeth are protected from age related damage and teeth are protected from discoloration, chipping and formation of cavities.

### BRIEF DESCRIPTION OF THE DRAWING

The invention will be more fully understood and further advantages will become apparent when reference is had to the following detailed description of the preferred embodiments of the invention and the accompanying drawing, in which:

FIG. 1 is a schematic representation of a Daytime and Nighttime composition delivery device.

### DETAILED DESCRIPTION OF THE INVENTION

The System of the subject invention comprises a complete morning-to-night protective regimen that restores and maintains youthful supporting structure to the teeth. Teeth need special care to keep them healthy and looking youthful. No matter how faithfully we brush and floss, teeth darken naturally over time, independent of food, coffee, or tobacco stains. They also chip, decay, and loosen. The breath becomes less fresh and gums bleed more easily. Teeth also dehydrate, causing tooth enamel to crack. The enamel peels away from the teeth and potholes develop at the gum line or chewing surfaces, causing the teeth to be more easily chipped and broken. Teeth also lose their ability to fight off microorganisms and other bacteria that cling to the teeth and get under the gums. Years of stress from dental repairs, bacteria, and oxidation also take their toll on teeth. Bleaching, bonding, and veneers can also create dehydration and stress. The System operates to combat these problems, which are closely related to aging of the mouth, gums, and teeth.

Generally stated, the present invention provides a system for preventing damage to oral tissue including mouth gums and teeth due to aging and includes (i) a system for application of oral hygiene products; (ii) compositions for use in the system; (iii) applicators especially tailored for use in applying the compositions to help fight the effects of aging on the mouth, gums and teeth on round-the-clock basis. The System provides complete daily care to help its users achieve a more youthful, healthy smile. It comprises three individual topical components: (a) a daytime whitener, moisturizer, and plaque remover; (b) a nighttime whitener, moisturizer, and plaque remover; and (c) an overnight power gel.

The System is suitable for people of all ages, since even people with young and healthy teeth and gums will benefit from the preventative care that is provided, in addition to the added benefits of fresher breath and whiter teeth. The System is preferably sold as a kit comprised of the following component parts: (i) one delivery device filled with the Daytime Composition; (ii) one delivery device filled with the Nighttime Composition; and (iii) one syringe filled with the Overnight Gel. Other component parts of the kit may include informational brochures and instructional leaflets. The kit, as sold, may contain a sufficient supply of the Daytime and Night-time Compositions, and the Overnight Gel, to facilitate refilling, and hence continuous use, of the delivery devices for a pre-selected time period, typically several months.

Daily use of the System combats the effects of aging on the teeth, gums and breath—all of which begins in the “tunnels”. Tunnels are the crevices between the teeth and gum line where the real problems start and get worse over time. These are the spots where food gets caught. They are the area where the gums bleed and become painful—where bad breath concentrates—where the worst stains begin—where the worst cavities are located—and where bone loss is worst. As years pass, these tunnels show signs of age before the rest of the mouth. That's where oral old age begins and where the power of the System starts to work and then continues on to combat the aging effects throughout the mouth.

Each of the three topical compositions comprises certain active ingredients, effective herbal ingredients and inert ingredients. As used herein, the term “active ingredients” or “active components” means ingredients which function as germicidal and/or anticaries agent in the mouth, gums or teeth. The term “effective herbal ingredients” or “effective herbal compounds” means ingredients from natural sources which function and have effects as an antimicrobial and/or antioxidant/anti-inflammatory agent in the mouth, gums and teeth. The compositions are tailored to be most effective for use during daytime, nighttime and overnight use respectively. The term “inert ingredients” or “inert components”, as used herein, refers to those ingredients in the compositions which are tailored to enhance dispersion of the components of the compositions, as well as enhancing the viscosity and delivery characteristics of the compositions and in maintaining a protective buffering capacity which mimicks the physiological pH of the oral tissues. For example, the daytime and nighttime compositions are free flowing so that each is distributed uniformly over the oral tissue. On the other hand, the overnight gel composition is a gel, which remains undisturbed on the gum, mouth and teeth surfaces while the user is sleeping, providing enhanced contact and absorption time.

The active components of the daytime and nighttime compositions, respectively, comprise xylitol and menthol. Xylitol has anticaries or cavity preventing properties and inhibits the growth of caries inducing bacteria. Replacement of sucrose and other products with xylitol prevents the growth of Streptococcus mutans bacteria, known to be a major contributor to the formation of dental cavities. The mutans act on sugar molecules creating acid, which has cavity forming ability. Xylitol, on the other hand, suppresses the acid formation capability of mutans bacteria and also prevents the growth of the bacteria, thereby providing an improved bacteria-free environment within the oral tissue including mouth, gums, teeth and oral tissue. Xylitol is a polyol and behaves very differently as compared to other sucrose base sweeteners. Xylitol is fermented by few oral bacteria and therefore suppresses the formation of plaque and acid formation. Xylitol has little or no cariogenic action in a rodent model inoculated with Streptococcus mutants, the key bacterium thought to be the leading cause of dental cavities and human in vivo experiments. The anticariogenic properties of xylitol do not only depend on an inhibition of bacterial metabolism, but in part on other mechanisms. Menthol chemically is an aromatic secondary alcohol obtained from the official oil of peppermint. Menthol possesses the general properties of peppermint oil somewhat intensified. It has germicidal, antiseptic and pronounced tonic properties. It has mild local anesthetic properties and counterirritant qualities. As a component of the daytime and nighttime compositions, it provides the soothing properties, and the mild germicidal actions to compliment the anticaries action of xylitol.

The effective herbal ingredients of the daytime and nighttime compositions, respectively, comprise green tea extract and goldenseal extract. The green tea extract blocks the production of oral bacteria associated with periodontal disease. Moreover, green tea extract has strong antioxidant properties and decreases oxidative DNA damage. Green Tea studies in medical research are numerous. Green tea and its constituents selectively induced apoptosis (programmed cell death) only in the oral carcinoma cell, such as submandibular lymph node metatases (cancers of the jaw/neck). The EGCG (epigallocatechin gallate) component of green tea was able to inhibit the growth and invasion of oral carcinoma cells. Recent human studies demonstrated that green tea consumption reduced oxidative DNA damage and free radical formation in smokers and nonsmokers. This property has been attributed to its antioxidant components known as green tea polyphenols. Green tea polyphenols have antimutagenic, anticarcinogenic, and antioxidant effects. A component of green tea, (−)epigallocatechin gallate (EGCG) inhibits the growth of cancer cells and metastasis. The production of toxic end metabolites of Porphyromonas gingivalis (a common pathogen in both periodontal and pulpal, i.e. gum and tooth, infections) and the production of butyric and propionic acids and phenylacetic acid by the bacteria is completely inhibited. The effects of green tea catechins on the proteinases produced by Porphyromonas gingivalis have been reported. These proteinases are associated with the breakdown of the periodontium. Catechin derivatives that included (−)-epigallocatechin gallate, (−)-epicatechin gallate, (−)-gallocatechin gallate and (−)-catechin gallate significantly inhibited the activity of Pophyromonas proteinases. Green tea also decreased the number of dysplasic lesions. The results suggested that green tea had inhibitory effects against oral carcinogenesis at the post-initiation stage and such inhibition may be related to the suppression of cell proliferation, induction of apoptosis, and the inhibition of angiogenesis. The major green tea constituents: (−)-epigallocatechin-3-gallate (EGCG), (−)-epicatechin-3-gallate (ECG) and (−) epigauocatechin (EGC) had effects on cell growth and DNA synthesis. All three compounds induced significant dose dependent inhibition in growth of the oral squamous carcinoma cells in culture. All three compounds induced dose-dependent inhibition of DNA synthesis. Goldenseal extract has anti-inflammatory properties and has anti-microbial properties against pathogens such as streptococcus mutans and fusobacterium nucleatum. Goldenseal extract is anti-inflammatory, anti-microbial and has “immune-strengthening” properties. The primary constituent of goldenseal is berberine and has nine other active ingredients. Berberine has antimicrobial activity against a broad array of pathogens including viruses and bacteria oral pathogens such as Streptococcus mutans and Fusobacterium nucleatum. The alkaloids in goldenseal have astringent activity that appears to contribute to its anti-inflammatory actions on mucous membranes, when treating sinusitis. These effective herbal ingredients in combination have an enhanced effect on reducing the microbial activity within the oral tissue and have healing properties that decrease the incidence of inflammation and lesions within the oral tissues.

The inert ingredients in the daytime and nighttime compositions comprise: taste and flavor enhancers including spearmint oil, stevia extract (a sugar free sweetener); mineral replacement additions including calcium chloride dehydrate, magnesium chloride; a buffer-system to maintain physiological pH including potassium phosphate monobasic, potassium phosphate dibasic, potassium chloride; bleaching or whitening agent including urea hydrogen peroxide; moisturizers including Arsalene 19, and thickeners including sodium carboxymethylcellulose and preserved water. The taste flavor enhancers improve the taste and flavor of the compositions. The mineral replacement additions provide an environment that strengthens the enamel and reduces its tendency to form cavities, and prevents discoloration and damage. The buffer-system is an acid neutralizer which maintains the physiological pH to prevent acidic damage to the oral tissues. The bleaching/whitening agent essentially works against harmful microbes present and removes surface stains on hard tissue. The thickeners adjust the viscosity so that the compositions can be easily delivered through the device and into the “tunnels” between the teeth and gum line.

The daytime composition has from 0.025% to 0.9% active ingredients with menthol and xylitol in a ratio of 1:36. The effective herbal components including green tea extract and goldenseal are included as a 0.06% concentration each. The inert ingredients include flavor enhancers, mineralizers, buffers and viscosity modifiers ranging from 0.006% to 5.000%.

The nighttime composition has from 0.025% to 0.9% active ingredients with menthol and xylitol in a ratio of 1:36. The effective herbal components including green tea extract and goldenseal are included as a 0.06% concentration each. The inert ingredients include flavor enhancers, mineralizers, buffers and viscosity modifiers ranging from 0.006% to 10.000%.

The active component of the Overnight gel composition comprises the biologically active extract of eucalyptus (eucalyptol). Eucalyptol has antibacterial effects against a number of different periodontopathic bacteria. It also has anti-inflammatory and analgesic (pain relieving) properties. Eucalyptus has antibacterial effects against a number of different periodontopathic bacteria including Bacillus subtilis and Streptococcus by the antimicrobial ingredient 1,8 cineol contained in eucalyptus. Eucalyptus oil has been shown to have antibacterial effects in vitro against the periodontopathic bacteria strains of Porphyromonas gingtvalis, Actinobacillus actionmycetemcomitans, Fusobacterium nucleatum, Streptococcus mutans and Streptococcus sobrinus. In this in vitro study, eucalyptus inhibited the growth in culture and showed significant adhesion-inhibiting activity against Streptococcus mutans. It also has anti-inflammatory and analgesic (pain relieving) properties. The effective herbal ingredients of the Overnight gel composition comprise Aloe Vera and Melissa officinalis (Lemon Balm) Oil. Aloe Vera extract is an effective tissue moisturizer and is an anti-inflammatory. It further has the ability to stop dental bleeding. Aloe Vera is an effective tissue moisturizer, is anti-inflammatory and has the ability to stop dental bleeding. Aloe vera gel is an effective tissue moisturizer with the following actions: is anti-inflammatory, is an enhancer of normal cell proliferation and pithelialization, is antibacterial and has analgesic properties. Aloe Vera has been shown to educe bleeding times and accelerate the healing process after surgeries. Melissa officinalis (Lemon Balm) oil is an effective anti-oxidant against selected oxidants, which could damage oral tissue. Topical application of Melissa is useful as prophylaxis (a preventative) against periodontal disease. Melissa officinalis (Lemon balm) is an effective anti-oxidant against selected oxidants, which could damage oral tissue. Melissa officinalis has activity against bacteria, fungi and yeasts and is effective against oral herpes simplex. Extracts of the leaves of Melissa officinalis were shown to inhibit the so-called “periodontopathic” bacteria. These are bacteria associated with and causing periodontal disease and include the Actinomyces species, Porphyromonas gingivalis and others. Topical application of Melissa officinalis could be useful as prophylaxis (a preventative) against periodontal disease.

The inert components of the Overnight gel include flavor and taste enhancing ingredients including Spearmint Oil, Stevia Extract (a non sucrose sweetener), Carbopol 934P NF thickener, a moisturizer retainer Trolamine NF (Triethanolamine) and Preserved Water.

The Overnight gel composition has the active component eucalyptol in concentration of 1.000%. Effective herbal ingredients Aloe Vera and Melissa Officinalis (Lemon Balm) Oil) are included in concentrations of 0.500% and 0.01% respectively in ratio of 50:1. The inert ingredients include flavor enhancers, viscosity modifiers and moisture retainers ranging from 0.500% to 1.5%.

A set of applicators are especially tailored to apply the Daytime Composition and the Nighttime Composition, respectively. The Overnight Gel is applied using a graduated syringe with a tapered applicator tip that fits into the tunnels between the teeth, that is, the gaps between the gum line and teeth surface. The Daytime composition applicator, Nighttime composition applicator and the Overnight Gel are organized in a time efficient and reliable manner in the system kit. The applicators are especially tailored for use in applying the compositions to help fight the effects of aging on the mouth, gums and teeth and to remove plaque on a round-the-clock basis. When used in connection with the present System, the applicators facilitate application of the oral hygiene products, providing twenty-four, seven protection against plaque formation and the effects of aging on the mouth, gums and teeth.

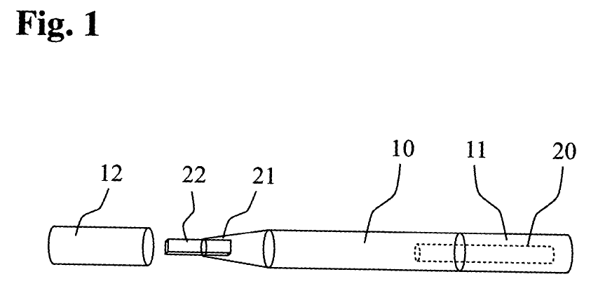

FIG. 1 shows a schematic diagram of the Daytime Composition and the Nighttime composition delivery device. The delivery device has a brush-like applicator tip 22 that fits in the “tunnels” between teeth and between gum line and teeth surface where it cleanses, moisturizes, whitens and helps plaque removal. At the same time, the applicator tip 22 massages and soothes the teeth and gums. The delivery device includes a pen-like tube 10 filled with the Daytime or Nighttime composition (not shown). At the first end of the tube 10 is the brush-like applicator tip 22, covered by a removable, press-fit cap 12. At the second end of the tube 10 is a ratchet device (not shown) that is operated by twisting a separate extension piece 11 attached to the second end of the tube. Each click of the ratchet moves a plunger 20 inside the tube 10 closer to the first end of the tube. This forces a small-predetermined amount of the composition through an aperture 21 and onto the brush-like applicator tip 22 for use. Each click releases an equal amount of composition, enabling application of the composition in controlled amounts. Preferably, the delivery device holds a total volume equal to about 500 “clicks”, of which about 20 “clicks” collectively comprise a daily dosage. The delivery device is compact and can be carried by the user during the day for more frequent use as a whitener, or to remove stubborn plaque and help stop bleeding gums. The delivery device is designed to fit most toothbrush holders. The overnight Gel is delivered via a syringe with a tapered applicator tip that fits in the “tunnels” of the mouth. A label on the syringe provides gradations to follow when applying the Overnight Gel. Preferably, the applicator for Overnight Gel composition delivers a daily dose indicated by a single line graduation of the graduated syringe.

The key features of the oral hygiene system to fight the effects of aging on the mouth gums and teeth include, in combination, the features set forth below:
1. The system comprising three topical compositional elements;
2. The first composition is a Daytime composition comprising active ingredients xylitol and menthol, together with effective herbal ingredients green tea extract and goldenseal extract together with inert ingredients that enhance the flavor and taste of the composition, a thickener to adjust the viscosity of the composition and a teeth whitener;
3. The second composition is a Nighttime composition comprising active ingredients xylitol and menthol, together with effective herbal ingredients green tea extract and goldenseal extract together with inert ingredients that enhance the flavor and taste of the composition, a thickener to adjust the viscosity of the composition and a larger quantity of teeth whitener;
4. The third composition is an Overnight gel comprising the active ingredient eucalyptol, the effective herbal ingredients aloe vera and Melissa officinalis (Lemon Balm) Oil together with the inert ingredients that enhance the flavor and taste of the composition, a thickener to adjust the viscosity of the composition and moisture retainer;
5. An applicator is provided with a brush like tip capable of delivering Daytime and Nighttime compositions to the tunnels between teeth and at the gum-teeth line from a barrel through the use of a ratchet mechanism that delivers a precise amount of the composition per click of the ratchet, wherein daily application requires approximately 20 clicks;
6. A graduated syringe applicator with a tapered tip is provided for delivering the Overnight gel;
   whereby the overall health of the oral tissues including mouth, gums and teeth are protected from age related damage and teeth are protected from discoloration, chipping and formation of cavities.

Having thus described the invention in rather full detail, it will be understood that such detail need not be strictly adhered to, but that additional changes and modifications may suggest themselves to one skilled in the art, all falling within the scope of the invention as defined by the subjoined claims.

What is claimed is:

1. An oral hygiene system for combating age related degradation of oral tissues including mouth, gums and teeth comprising:
a. a first topical application composition for use as a Daytime composition;
b. a second topical application composition for use as a Nighttime composition;
c. each of said Daytime and Nighttime compositions, respectively, comprising active ingredients of Xylitol and menthol;
d. each of said Daytime and Nighttime compositions, respectively, comprising effective herbal ingredients of green tea extract and goldenseal extract;
e. each of said Daytime and Nighttime compositions, respectively, comprising inert ingredients including flavor and taste enhancers, mineral replacement additions, thickeners, moisturizers, buffers, whitening agents and preserved water;
f. said Nighttime composition having a higher concentration of said whitening agent than said Daytime composition;
g. a third topical application composition for use as a Overnight gel composition;
h. said Overnight gel composition comprising active ingredient of Eucalyptol;
i. said Overnight gel composition comprising effective herbal ingredients of aloe vera and Melissa officinalis (Lemon Balm) Oil;
j. said Overnight gel composition comprising inert components including flavor and taste enhancers, thickener to adjust the viscosity of said composition, moisture retaining agents and preserved water;
k. said Daytime and Nighttime compositions applied to oral tissue and tunnels of teeth with an applicator provided with a brush tip and a ratchet ‘click’ mechanism to deliver precise quantity of the composition per click;
l. said Overnight gel delivered to oral tissue and teeth by a graduated syringe with tapered tip deigned to fit into the tunnels between teeth and at gum-line;
whereby the overall health of the oral tissue including mouth, gums, and teeth are protected from age related damage and teeth are protected from discoloration, chipping and formation of cavities.

2. The oral hygiene system as recited by claim 1, wherein said Daytime composition has from 0.025% to 0.9% said active ingredients with menthol and xylitol in a ratio of 1:36.

3. The oral hygiene system as recited by claim 1, wherein said Daytime composition has said effective herbal ingredients in concentrations of 0.06% green tea extract and 0.06% goldenseal.

4. The oral hygiene system as recited by claim 1, wherein said Daytime composition has said inert ingredients including flavor enhancers, mineralizers, buffers, whitening agent, and viscosity modifiers ranging from 0.006% to 5.000%.

5. The oral hygiene system as recited by claim 1, wherein said Nighttime composition has from 0.025% to 0.9% active ingredients with menthol and xylitol in a ratio of 1:36.

6. The oral hygiene system as recited by claim 1, wherein said Nightime composition has said effective herbal ingredients in concentrations of 0.06% green tea extract and 0.06% goldenseal.

7. The oral hygiene system as recited by claim 1, wherein said Nighttime composition comprises said inert ingredients including flavor enhancers, mineralizers, buffers, whitening agent, and viscosity modifiers ranging from 0.006% to 10.000%.

8. The oral hygiene system as recited by claim 1, wherein said Daytime composition has from 2% to 8% of said whitening agent.

9. The oral hygiene system as recited by claim 8, wherein said Daytime composition has 5% of said whitening agent.

10. The oral hygiene system as recited by claim 1, wherein said Nighttime composition has from 4% to 16% of said whitening agent.

11. The oral hygiene system as recited by claim 10, wherein said Nighttime composition has 10% of said whitening agent.

12. The oral hygiene system as recited by claim 1, wherein said taste enhancers of said Daytime and Nighttime compositions, respectively, comprise spearmint oil and stevia extract.

13. The oral hygiene system as recited by claim 1, wherein said mineralizers of said Daytime and Nighttime compositions, respectively, comprise calcium chloride dehydrate and magnesium chloride.

14. The oral hygiene system as recited by claim 1, wherein said whitening agent of said Daytime and Nighttime compositions, respectively, comprise urea hydrogen peroxide.

15. The oral hygiene system as recited by claim 1, wherein said thickening agent of said Daytime and Nighttime compositions, respectively, comprise sodium carboxymethylcellulose and preserved water.

16. The oral hygiene system as recited by claim 1, wherein said moisturizer of said Daytime and Nighttime compositions, respectively, comprises Arsalene 19.

17. The oral hygiene system as recited by claim 1, wherein said taste enhancer of said Overnight Gel composition comprises Spearmint Oil and Stevia Extract.

18. The oral hygiene system as recited by claim 1, wherein said thickener of said Overnight Gel composition comprises Carbopol 934P NF.

19. The oral hygiene system as recited by claim 1, wherein said moisturizer of said Overnight Gel composition comprises Trolamine NF (Triethanolamine) and preserved water.

20. The oral hygiene system as recited by claim 1, wherein said applicator for Daytime and Nighttime compositions, respectively, has a capacity of about 500 clicks.

21. The oral hygiene system as recited by claim 1, wherein said applicator for Daytime and Nighttime compositions, respectively, delivers a, daily dose of about 20 clicks.

22. The oral hygiene system as recited by claim 1, wherein said applicator for Overnight Gel composition delivers a daily dose indicated by a single line graduation of said graduated syringe.

23. An oral hygiene composition for combating age related degradation of oral tissues including mouth, gums and teeth, comprising:
a. a topical application composition for use as a Daytime composition;
b. said Daytime composition comprising active ingredients of Xylitol and menthol;
c. said Daytime composition comprising effective herbal ingredients of green tea extract and goldenseal extract;
d. said Daytime composition comprising inert ingredients including flavor and taste enhancers, mineral replacement additions, thickeners, moisturizers, buffers, whitening agents and preserved water;
e. said Daytime composition applied to oral tissue and tunnels of teeth with an applicator provided with a brush tip and a ratchet ‘click’ mechanism to deliver precise quantity of the composition per click;
whereby the overall health of the oral tissue including mouth, gums, and teeth are protected from age related damage and teeth are protected from discoloration, chipping and formation of cavities.

24. An oral hygiene composition for combating age related degradation of oral tissues including mouth, gums and teeth comprising:
a. a topical application composition for use as a Nighttime composition;
b. said Nighttime composition comprising active ingredients of Xylitol and menthol;
c. said Nighttime composition comprising effective herbal ingredients of green tea extract and goldenseal extract;
d. said Nighttime composition comprising inert ingredients including flavor and taste enhancers, mineral replacement additions, thickeners, moisturizers, buffers, whitening agents and preserved water;
e. said Nighttime composition applied to oral tissue and tunnels of teeth with an applicator provided with a brush tip and a ratchet ‘click’ mechanism to deliver precise quantity of the composition per click;
whereby the overall health of the oral tissue including mouth, gums, and teeth are protected from age related damage and teeth are protected from discoloration, chipping and formation of cavities.

25. An oral hygiene composition for combating age related degradation of oral tissues including mouth, gums and teeth comprising:
a. a topical application composition for use as a Overnight gel composition;
b. said Overnight gel composition comprising active ingredient of Eucalyptol;
c. said Overnight gel composition comprising effective herbal ingredients of aloe vera and Melissa officinalis (Lemon Balm) Oil;
d. said Overnight gel composition comprising inert components including flavor and taste enhancers, thickener to adjust the viscosity of said composition, moisture retaining agents and preserved water;
e. said Overnight gel delivered to oral tissue and teeth by a graduated syringe with tapered tip deigned to fit into the tunnels between teeth and at gum-line;
whereby the overall health of the oral tissue including mouth, gums, and teeth are protected from age related damage and teeth are protected from discoloration, chipping and formation of cavities.
